(19) United States
(12) Patent Application Publication
(10) Pub. No.: US 2004/0234500 A1
(43) Pub. Date: Nov. 25, 2004

(54) Method for inducing proliferation of retinal stem cells

(76) Inventors: Emma E. Moore, Seattle, WA (US); Thomas Reh, Seattle, WA (US)

Correspondence Address: Michelle I Johnson, ZymoGenetics Inc, 1201 Eastlake Avenue East, Seattle, WA, 98102, US

(21) Appl. No.: 10/472,916

(22) PCT Filed: Mar. 18, 2002
(86) PCT No.: PCT/US02/07967

Related U.S. Application Data
(60) Provisional application No. 60/278,891, filed on Mar. 26, 2001.

(51) Int. Cl. A61K 45/00; A61K 38/20

(52) U.S. Cl. 424/85.2; 424/93.7

(57) ABSTRACT

The present invention relates to a method for promoting the proliferation of retinal stem cells using IL-17B. IL-17B is put into a cell culture medium containing retinal stem cells to promote the proliferation and/or differentiation of retinal stem cells. The retinal stem cells can then be transplanted into the retina to promote the growth of the photoreceptor cells, the rods and the cones. Also IL-17B can be administered directly into the retina to promote the proliferation and/or differentiation of the retinal stem cells.

### BACKGROUND OF THE INVENTION

Vision is one of the most important special senses in humans. Light enters the eye and impinges on photoreceptors of a specialized epithelium, the retina. The photoreceptors include rods and cones. Rods have low thresholds for detecting light and operate best under conditions of reduced lighting (scoptic vision). However, rods neither provide well-defined visual images nor contribute to color vision. Cones, by contrast, are not as sensitive as rods to light and so operate best under daylight conditions (photopic vision). Cones are responsible for high visual acuity and color vision.

Information processing within the retina is performed by retinal interneurons, and the output signals are carried to the brain by the axons of retinal ganglion cells. Fetal and adult retinal stem cells give rise to all the various cell types in the retina including a) the rod and the cone photoreceptors, b) the horizontal, bipolar, and amacrine interneurons, c) the ganglion projection neurons, and d) the Muller glia cells.

Degenerative diseases of the retina often result in blindness due to the destruction of the rods or cones. Retinal stem cell therapy has been developed in which retinal stem cells are harvested from the patient grown and expanded in culture and reintroduced into the retina in an attempt to promote regeneration of the rods and cones. Growth factors that have been used in culture to promote proliferation of the retinal stem cells include a) transforming growth factor alpha (TGF-α) and epidermal growth factor (EGF), b) fibroblast growth factor (FGF), c) TGF-β 2 & 3, and d) sonic hedgehog (shh). While these growth factors are useful, there is still a need to discover additional agents to promote the proliferation and differentiation of retinal stem cells into photoreceptor rods or cones.

### DESCRIPTION OF THE INVENTION

The present invention fills this need by providing for a method of promoting the proliferation of retinal stem cells comprising bringing IL-17B into contact with retinal stem cells. Retinal stem cells can be grown in culture into which IL-17B is added and re-implanted into a patient's retina to produce functioning rods and cones of the retina. Alternatively, the IL-17B can be administered directly into retina.

The teachings of all of the references cited in the present specification are incorporated in their entirety herein by reference.

### Definitions

The term “effective amount” as used herein regarding the effective amount of IL-17B administered in accordance with the present invention means an amount of IL-17B that causes proliferation of retinal stem cells. The effective amount of IL-17B or IL-17 to be administered is from 0.1 μg to 100 μg of IL-17B or IL-17 per kilogram of body weight per day. More preferably, the effective amount is from 1 μg to 500 μg of IL-17B or IL- 17 per kilogram of body weight. IL-17B should be administered daily until the symptoms of neuropathy dissipate. If the retinal stem cells are grown in culture, the concentration of IL-17B in the culture medium should be at least 100 ng/ml.

IL-17B (formerly called ‘Zcyto7’) and a method for making IL-17B polypeptides have been disclosed in International Patent Application No. PCT/US98/08212, Publication No. WO 98/49310.

### Introduction

The present invention is based upon the discovery that IL-17B or IL-17 can induce the proliferation and/or differentiation of retinal stem cells. IL-17B can be used to treat many ocular disorders in which retinal neurons have degenerated, such as macular degeneration and glaucoma. Age-related macular degeneration is the leading cause of blindness in the United States. Currently, there is no satisfactory treatment. In promoting the proliferation of retinal stem cells, one can administer IL-17B directly into the retina or by a gene therapy modality to stimulate the growth of endogenous stem cells. Secondly, retinal stem cells can be removed from the patient and IL-17B can be used to stimulate the growth of retinal stem cells in vitro, and then transplant the stem cells back into the retina of the patient.

Those skilled in the art will recognize that the sequences disclosed in SEQ ID NOs: 1, and 2 represent a single allele of the human IL-17B. One can clone allelic variants of these sequences by probing cDNA or genomic libraries from different individuals according to standard procedures.

### Modes of Administration

In general, pharmaceutical formulations will include an IL-17B protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., (Mack Publishing Co., Easton, Pa., 19th ed., 1995). In a culture medium. in which retinal stem cells are growing, the IL-17B should be present at a concentration of at least 100 ng/ml. If the IL-17B is administered directly into the retina, the therapeutic doses will generally be in the range of 0.1 to 100 μg/kg of patient weight, with the exact dose determined by the clinician according to accepted standards determination of dose is within the level of ordinary skill in the art. The proteins may be administered for acute treatment, over one week or less, often over a period of one to three days or may be used in chronic treatment, over several months or years.

### Nucleic Acid-Based Therapeutic Treatment

IL-17B can be also administered to a retinal stem cell by means of gene therapy. In one embodiment, a gene encoding an IL-17B polypeptide is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector [Kaplitt et al., Molec. Cell. Neurosci. 2: 320-330 (1991)], an attenuated adenovirus vector, such as the vector described by Stratford-Perricaudet et al., J. Clin. Invest. 90:626-630 (1992), and a defective adeno-associated virus vector [Samulski et al., J. Virol., 61:3096-3101 (1987); Samulski et al. J. Virol., 63:3822-3828 (1989)].

In another embodiment, the gene can be introduced into a retinal stem cell by means of a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995 by Dougherty et al. and Blood, 82:845 (1993).

Alternatively, the vector can be introduced by lipofection in vivo using liposomes. Synthetic cationic lipids can be used to prepare liposomes for in vivo transfection of a gene encoding a marker [Felgner et al., Proc. Natl. Acad. Sci. USA, 84:7413-7417 (1987); see Mackey et al., Proc. Natl. Acad. Sci. USA, 85:8027-8031 (1988)]. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

It is possible to remove the retinal stem cells from the body and introduce the vector as a naked DNA plasmid and then re-implant the transformed cells into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter [see, e.g., Wu et al., J. Biol. Chem., 267:963-967 (1992); Wu et al., J. Biol. Chem., 263:14621-14624 (1988)].

### EXAMPLE 1

### Cloning of IL-17B

IL-17B was identified from expressed sequence tag (EST) 582069 (SEQ ID NO: 3) by its homology to Interleukin-17. The EST582069 cDNA clone was obtained from the IMAGE™ consortium Lawrence Livermore National Laboratory through Genome Systems, Inc. The cDNA was supplied as an agar stab containing E. coli transfected with the plasmid having the cDNA of interest and then streaked out on an LB 100 μg/ml ampicillin and 100 μg/ml methicillin plate. The cDNA insert in EST582069 was sequenced. The insert was determined to be 717 base pairs long with a 180 amino acid open reading frame and a 22 amino acid signal peptide.

### EXAMPLE 2

### Construction of IL-17B Expression Vectors

A 473 bp IL-17B PCR DNA fragment was generated with 1 μl of a dilution of the EST582069 plasmid prep of Example 2 and 20 picomoles (pm) of primer SEQ ID NO: 4 and 20 pm primer SEQ ID NO: 5. The digested reaction mixture was electrophoresed on a 1% TBE gel; the DNA band was excised with a razor blade and the DNA was extracted from the gel with the Qiaquick<< Gel Extraction Kit (Qiagen). The excised DNA was subcloned into plasmid nfpzp9, which had been cut with Bam and Xho. Nfpzp9 is a mammalian cell expression vector comprising an expression cassette containing the mouse metallothionein-1 promoter, a sequence encoding the tissue plasminogen activator (TPA) leader, then multiple restriction sites. These were followed by the human growth hormone terminator, an E. coli origin of replication and a mammalian selectable marker expression unit containing the SV40 promoter, enhancer and origin of replication, a dihydrofolate reductase gene (DHFR) and the SV40 terminator.

IL-17B was purified by means of affinity chromatography using anti-IL-17B antibodies.

### EXAMPLE 3

### Cloning of Murine IL-17B

Mouse IL-17B was identified from an expressed sequence tag (EST) 660242 (SEQ ID NO: 8). EST660242 cDNA clone was obtained from the IMAGE consortium Lawrence Livermore National Laboratory through Genome Systems, Inc. The cDNA was supplied as an agar stab containing E. coli transfected with the plasmid having the cDNA of interest and then streaked out on an LB 100 μg/ml ampicillin, 25 μg/ml methicillin plate. The cDNA insert in EST660242 was sequenced. The insert was determined to be 785 base pairs with an open reading frame of 180 amino acids and a putative 20 amino acid signal peptide. The sequences are defined by SEQ ID NO: 7 and SEQ ID NO: 6.

### EXAMPLE 4

### Proliferation of Retinal Stem Cells in Culture

Retinal stem cells were obtained from the retina of E17-18 rat embryos and grown in culture. Preliminary results indicate that human recombinant IL-17B stimulates the growth of retinal stem cells. The cells spread out on the substrate within one day, and the IL-17B-treated cells appeared to proliferate more rapidly than the control cells. We verified that IL-17B stimulated the proliferation of these cells by using an antibody that recognizes a protein present in M-phase cells (phosphohistone3). We found many more cells labeled with phospho-histone3 antibody in the culture containing the IL-17B.

We claim:

1. A method for inducing the proliferation and/or differentiation of retinal stem cells comprising bringing the retinal stem cells into contact with interleukin-17B (IL-17B).

2. The method of claim 1 wherein the IL-17B polypeptide is selected from the group consisting of SEQ ID NOs: 2, 7, and 9-28.

3. The method of claim 1 wherein the retinal stem cells are grown in a culture medium.

4. The method of claim 3 wherein the retinal stem cells are implanted into the retina of a mammal after the stems cells have come into contact with IL-17B.

5. A method for inducing the proliferation and/or differentiation of retinal stem cells comprising administering IL-17B into the retina.


SEQUENCE LISTING (28 sequences)

SEQ ID NO: 1 - DNA, 736 bp, Homo sapiens
  CDS (57)...(596)
gaattcggcacgaggaggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagccagaaaaaaaaaaaaaaaaaaaaattcctgcggccgc

SEQ ID NO: 2 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 3 - DNA, 397 bp, Homo sapiens
  variation 10: n = A, T, C, or G
aggcgggcanagctgcaggctgaccttgcagcttggcggaatggactggcctcacaacctgctgtttcttcttaccatttccatcttcctggggctgggcagccaggagccccaaaagcaagaggaaggggcaagggcggcctgggcccntggcctggcctcaccaggtgccactggacctggtgtcacggatgaaaccgtatgcccgcatggaggagtatgagaggaacatcgaggagatggtggcccagctgaggaacagctcanaagctggcccagagaaagtgtgaggtcaacttgcagctgtggatgtccaacaagaaggagcctgtctcccttggggctacaagcatcaaccaccgaccccagccgtatccccgtgggaccttgccgggac

SEQ ID NO: 4 - DNA, 18 bp, Homo sapiens
ttaccatttccatcttcc

SEQ ID NO: 5 - DNA, 18 bp, Homo sapiens
cccttcctcttgcttttg

SEQ ID NO: 6 - DNA, 692 bp, Mus musculus
  CDS (50)...(589)
ggggttcctggcgggtggcagctgcgggcctgccgcctgacttggtgggatggactggccgcacagcctgctcttcctcctggccatctccatcttcctggcgccaagccacccccggaacaccaaaggcaaaagaaaagggcaagggaggcccagtcccttggcccctgggcctcatcaggtgccgctggacctggtgtctcgagtaaagccctacgctcgaatggaagagtatgagcggaaccttggggagatggtggcccagctgaggaacagctccgagccagccaagaagaaatgtgaagtcaatctacagctgtggttgtccaacaagaggagcctgtccccatggggctacagcatcaaccacgaccccagccgcatccctgcggacttgcccgaggcgcggtgcctatgtttgggttgcgtgaatcccttcaccatgcaggaggaccgtagcatggtgagcgtgccagtgttcagccaggtgccggtgcgccgccgcctctgtcctcaacctcctcgccctgggccctgccgccagcgtgtcgtcatggagaccatcgctgtgggttgcacctgcatcttctgagccaaccaccaacccggtggcctctgcaacaaccctccctccctgcacccactgtgaccctcaaggctgataaacagtaaacgctgttctttgtaaagga

SEQ ID NO: 7 - protein, 179 aa, Mus musculus
MDWPHSLLFLLAISIFLAPSHPRNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCI

SEQ ID NO: 8 - DNA, 497 bp, Mus musculus
ggggttcctggcgggtggcagctgcgggcctgccgcctgacttggtgggatggactggccgcacagcctgctcttcctcctggccatctccatcttcctggcgccaagccacccccggaacaccaaaggcaaaagaaaagggcaagggaggcccagtcccttggcccctgggctcatcaggtgccgctggacctggtgtctcgagtaaagccctacgctcgaatggaagagtatgagcggaaccttggggagatggtggcccagctgaggaacagctccgagccagccaagaagaaatgtgaagtcaatctacagctgtggttgtccaacaagaggagcctgtccccatggggctacagcatcaaccacgaccccagccgcatccctgcggacttgcccgaggcgcggtgcctatgtttgggttgcgtgaatcccttcaccatgcaggaggaccgtagcatggtgagcgtgccagtgttcagccaggtgccggtgcg

SEQ ID NO: 9 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 10 - protein, 160 aa, Homo sapiens
QPRAPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 11 - protein, 160 aa, Homo sapiens
QPRSPKAKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 12 - protein, 160 aa, Homo sapiens
QPRSPKSKRKGQGRPAPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 13 - protein, 160 aa, Homo sapiens
QPRSPKSKRKGQGRPGPLAPGPHQVPLDLVARMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 14 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRVVMETIAVGCTCIF

SEQ ID NO: 15 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRLVMETIAVGCTCIF

SEQ ID NO: 16 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRFVMETIAVGCTCIF

SEQ ID NO: 17 - protein, 160 aa, Homo sapiens
QPRSPKSKRKGQGRPGPLAPGPHQVPLDLVGRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 18 - protein, 160 aa, Homo sapiens
QPRSPKSKRKGQGRPSPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 19 - protein, 160 aa, Homo sapiens
QPRSPKVKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 20 - protein, 160 aa, Homo sapiens
QPRVPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 21 - protein, 158 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 22 - protein, 154 aa, Homo sapiens
SKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 23 - protein, 151 aa, Homo sapiens
KGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 24 - protein, 160 aa, Homo sapiens
HPRNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 25 - protein, 158 aa, Homo sapiens
RNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 26 - protein, 153 aa, Homo sapiens
KRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 27 - protein, 128 aa, Homo sapiens
MKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 28 - protein, 157 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCI